(19) United States
(12) Patent Application Publication
(10) Pub. No.: US 2011/0313522 A1
(43) Pub. Date: Dec. 22, 2011

(54) Pseudophakic Accommodating Intraocular Lens

(75) Inventor: Anna S. Hayes, Newton Centre, MA (US)

(73) Assignee: Anew Optics, Inc., Newton Centre, MA (US)

(21) Appl. No.: 13/113,815

(22) Filed: May 23, 2011

Related U.S. Application Data
(63) Continuation-in-part of application No. 12/626,473, filed on Nov. 25, 2009.
Application No. 12/626,473 is a continuation-in-part of application No. 12/626,459, filed on Nov. 25, 2009.
(60) Provisional application No. 61/118,085, filed on Nov. 26, 2008.
(60) Provisional application No. 61/118,076, filed on Nov. 26, 2008.
(60) Provisional application No. 61/347,083, filed on May 21, 2010.
(60) Provisional application No. 61/381,784, filed on Sep. 10, 2010.

(51) Int. Cl. A61F 2/16

(52) U.S. Cl. 623/6.43

(57) ABSTRACT

The invention is directed to an assembly comprising a haptic for fixation to, and manufacture in conjunction with, an intraocular lens to be implanted in the natural lens capsule of an eye. The haptic of the invention comprises a continuous ribbon forming an essentially oblong shape having anterior and posterior portions relative to the elliptical center of the haptic, wherein the ribbon loop includes two or more essentially congruent ribbon arches in each portion, and each ribbon arch has a natural index of curvature with an inner and outer edges. designed to expand the eye capsule and put tension on the zonules of the eye. The ribbon affixes to the lens on each side of the optic edge at a point or a series of points that provides suitable centration and stability of the optic, and to suspend the optic in the open capsular space. The material of the haptic is preferably somewhat flexible, and elastic, so as to provide a constant, positive force on the capsule throughout all phases of accommodation, thereby preserving tension of the zonules and allowing the capsule to change shape naturally. The haptic ribbons may be solid or of an open work structure to increase the amount of hydration available to the lens capsule. A secondary haptic ribbon, affixed to a plano optical plate, may be located on the posterior capsular surface and oriented so that the haptic arms extend through the capsular prime meridian to the anterior capsular surface at a 90° angle from the anterior haptic ribbons, thus providing for a capsular configuration as natural as possible, yet associated with an intraocular lens that may be inserted through an incision of less than 3 millimeters.

### REFERENCE TO RELATED APPLICATIONS

This application claims priority to U.S. Provisional Application No. 61/347,083 entitled “Pseudophakic Accommodating Intraocular Lens” filed May 21, 2010, and U.S. Provisional Application No. 61/381,784 entitled “Pseudophakic Accommodating Intraocular Lens” filed Sep. 10, 2010, and is a continuation-in-part of U.S. application Ser. No. 12/626,473 entitled “Haptic Devices for Intraocular Lens” filed Nov. 25, 2009 which claims priority to U.S. Provisional Application No. 61/118,085 entitled “Haptic Devices for Intraocular Lens” filed Nov. 26, 2008, and of U.S. application Ser. No. 12/626,459 entitled “Intraocular Lens Optic” filed Nov. 25, 2009 which claims priority to U.S. Provisional Application No. 61/118,076 entitled “Intraocular Lens Optic” filed Nov. 26, 2008, all of which are incorporated by reference in their entirety.

### BACKGROUND OF THE INVENTION

1. Field of the Invention

The invention is directed to haptic devices for intraocular lenses that provide increased comfort and performance to a patient. In particular, the invention is directed to haptic devices and designs for positioning the intraocular lens appropriately within the natural capsule of the eye after removal of the natural, crystalline lens, while maintaining, as much as possible, the natural configuration of the lens capsule. Specifically, the invention, along with its various iterations, is designed to provide suitable degrees of focal flexibility, or accommodation, when used in conjunction with one or ore monofocal optics, and, in certain instances, is designed to mitigate the onset of post-surgical conditions, specifically Posterior Capsular Opacification.

2. Description of the Background

An intraocular lens (IOL) is an implanted lens in the eye, usually replacing the existing crystalline lens because it has been clouded over by a cataract, or as a form of refractive surgery to change the eye's optical power. The whole device usually comprises a small plastic lens with plastic side struts, called haptics, to hold the lens in place within the lens capsule inside the eye. Haptics also form the means of attachment of lenses to other areas of the eye, including the anterior chamber angle or sulcus, the iris, or the posterior chamber ciliary sulcus. IOLs were traditionally made of an inflexible material (e.g. PMMA) though this largely been superseded by the use of flexible materials. Most IOLs fitted today are fixed monofocal lenses matched to distance vision. However, other types are available, such as multifocal IOLs which provide the patient with multiple-focused vision at far and reading distance, toric IOLs to correct for astigmatisms, and adaptive IOLs which provide the patient with limited visual accommodation.

Intraocular lenses have been used since 1999 for correcting larger errors in myopic (near-sighted), hyperopic (far-sighted), and astigmatic eyes. A phakic intraocular lens (PIOL) is inserted into the eye without removal of the natural crystalline lens. An aphakic IOL (that is, not PIOLs) is used for correction of higher refractive errors (especially substantial hyperopia), and is implanted via Clear Lens Extraction and Replacement (CLEAR) surgery. During CLEAR, the crystalline lens is extracted and an IOL replaces it in a process that is very similar to cataract surgery.

Once implanted, CLEAR IOL procedures have three major benefits. First, they represent an alternative to LASIK, a form of eye surgery that may not work for people with serious vision problems. Second, effective IOL implants may eliminate the need for glasses or contact lenses post-surgery. Third, a CLEAR recipient will not develop cataracts, as the natural lens has been removed, although most current CLEAR recipients risk onset of PCO (posterior capsule opacification) and/or ACO (anterior capsule opacification) in some form. The disadvantage to CLEAR is that the eye's ability to change focus (accommodate) may have been reduced or eliminated, depending on the kind of lens implanted.

While significant advances have been made in the optical quality of aphakic lenses, most lenses currently made have an overall optical thickness of one millimeter or greater at the center optical focal point (e.g. see U.S. Pat. No. 4,363,142). In the late 1990's, two patents were applied for and subsequently issued for lens optics significantly thinner than the afore-referenced lens patents (U.S. Pat. Nos. 6,096,077 and 6,224,628). Although improved, the extreme thinness of the lens manufactured in accordance with U.S. Pat. No. 6,096,077 caused some minor distortions of the optic once in the eye, while the lens manufactured in accordance with the manufacturing methods of U.S. Pat. No. 6,224,628 was poured of molded silicone and did not provide the desired visual acuity. Each of the preceding patents is specifically incorporated by reference.

The natural lens is contained in a membrane known as the lens capsule. The artificial lenses are fixedly attached within the eye, either by stitching to the iris, or by some supporting means or arms attached to the lens; in all cases the parts of the lens that allow for fixation to the eye are categorized as haptics.

Several intraocular lenses designed for implant in the anterior chamber feature haptics with feet which support the lens in order to avoid the need for clips or sutures to secure the lens to the iris. A wide variety of lenses has been developed with up to four haptics. The haptics are linked to the lens body so that the support structure should not deflect freely of the lens body. A variety of shapes and geometries for the lens supporting elements, or haptics, has been disclosed and described in the literature (e.g. see U.S. Pat. No. 4,254,510; U.S. Pat. No. 4,363,143; U.S. Pat. No. 4,480,340; U.S. Pat. No. 4,504,981; U.S. Pat. No. 4,536,895; U.S. Pat. No. 4,575,374; U.S. Pat. No. 4,581,033; U.S. Pat. No. 4,629,460; U.S. Pat. No. 4,676,792; U.S. Pat. No. 4,701,181; U.S. Pat. No. 4,778,464; U.S. Pat. No. 4,787,902; U.S. Pat. No. Re. 33,039; U.S. Pat. No. 4,872,876; U.S. Pat. No. 5,047,052; U.K. Patent No. 2,165,456—all of which are specifically incorporated by reference).

Despite the advances, there remain problems with intraocular implants. For example, when an intraocular lens is inserted in the eye, an incision is made in the cornea or sclera. The incision may cause the cornea to vary in thickness, leading to an uneven surface which can cause astigmatism. The insertion of a rigid lens through the incision, even with compressible haptics, requires an incision large enough to accommodate the rigid lens (typically at least 6 mm), and carries with it the increased risk of complications, such as infection, laceration of the ocular tissues, and retinal detachment. Deformable intraocular lenses made from polymethylmethacrylate (e.g. “PMMA”), polysulfone, silicone or hydrogel may be inserted through a smaller incision. Current surgical practices call of intraocular lenses that can be safely inserted through an incision of less than 3 mm, preferably less than 2.8 mm, as practice has demonstrated better patient outcomes with smaller incisions.

It is preferred that the intraocular lens be capable of insertion through a small incision. U.S. Pat. No. 4,451,938 shows an intraocular lens in which the lens body is made in two pieces so that each piece may be inserted through the incision separately and then joined by dowels after insertion in the eye. U.S. Pat. No. 4,769,035 discloses a foldable lens which may be inserted through an incision about 3.5 mm in length.

The haptics of intraocular lenses that are implanted into the posterior chamber may attach to the ciliary sulcus or be positioned in the equator of the capsule. In each case the haptics must be the correct size to ensure proper anchoring. If the haptics are too short for the capsule, the lens can dislodge or rotate in the eye possibly causing intraocular trauma or visual anomalies, and the patient may require additional surgery. Additionally, haptics that are too short for the capsule do not allow the lens to provide the patient with any desired or designed focal flexibility (that is, accommodation). If the haptic is too long for the capsule, the lens can angle either posteriorly or anteriorly at a greater angle than designed, which will alter the position of the optic and induce unwanted refractive errors.

U.S. Pat. Nos. 5,258,025 and 5,480,428 describe a lens surrounded by a sheet-like “positioner” having projections called “supporting elements either at the four corners of or continuously around the positioner, the supporting elements being 0.3 mm long and 0.01 to 0.05 mm thick (7″a and 7″b of FIG. 3 of the '025 patent, 18 of the '428 patent). However, the lens is for implantation in the posterior chamber, the lens of the '428 actually having a length short enough to “float.” In addition, the sheet-like nature of the positioner prevents independent deflection of the feet in response to forces applied by the eye.

In addition, the lens may place a greater or lesser degree of force on the haptic feet as the lens is compressed, depending upon construction of the lens. Since the amount of pressure for a given surface area is proportional to the force, it is desirable to decrease or distribute the amount of force placed on the haptic feet in order to diminish the force applied by the feet on the respective membranes of the eye to which the lens is most proximate. For example, in the case of anterior chamber IOLs, the trabecular meshwork; in the case of sulcus fizated lenses, the ciliary processes; in the case of iris fizated lenses, the iris, and in the case of intracapsular lenses, the ciliary body and the zonules. In the case of the Lens of the invention, this goal is achieved by contouring the haptics such that they are in contiguous contact with the lens capsule, conforming to the natural contours of the capsule and thereby reducing particular points of pressure from the accommodative equation.

The latest science is developing an understanding of the importance of maintaining the natural physics of the eye. In current IOL designs, most IOLs are essentially two dimensional, which has the effect of stretching the capsule toward the equator of the lens, and causing the posterior capsule to move anteriorly and the anterior capsule to move posteriorly. Whereas the aqueous humor will essentially equalize the pressure of the anterior capsule, displacing the posterior capsule from its natural configuration may cause negative pressure, or vacuum, on the vitreous, which may increase the risk of post-surgical retinal detachment and/or macular degeneration. Additionally, deforming the natural configuration of the lens capsule has been demonstrated to cause capsular fibrosis, thus increasing the risk of PCO, ACO, and exacerbating loss over time of accommodative powers. Also, deforming the capsule significantly may affect the zonules, which are fixed-length fibers that connect the capsule to the ciliary body and whose connection points extend throughout a zone from a point on the anterior capsule to a point on the posterior capsule. Deformation of may cause these zonules to break, thus risking entire displacement of the capsule itself and potential complete loss of vision in the patient.

The act of surgically removing the natural lens and replacing it with an intraocular lens can give rise to certain other possible conditions that may reduce the patient's ability to see clearly over a protracted period of time and/or the extent of focal accommodation that can be provided to the patient, and may change the actual positioning of the replacement lens in the eye from its original design. In particular, ophthalmologists have observed that the lens capsule will tend to contract over time. This is in part attributable to the fact that the replacement lens rarely occupies the entire lens capsule, and most lenses tend to flatten out the capsule, thus allowing the anterior and posterior surfaces of the lens capsule to adhere come into contact with each other, and causing the capsule to fibrose and shrink around the IOL. All these will necessarily diminish the effectiveness of any lens claiming to offer focal accommodation. It is possible that increased circulation of the aqueous humor can preserve the suppleness of the natural lens capsule, and preventing contact between the capsular surfaces may prevent capsular adhesions.

Some physicians have advocated the use of capsular tension rings to prevent capsular shrinkage. However, these rings, which are placed inside the lens capsule at the equator, do not allow the ciliary body to influence the dimensions of the lens so as to provide for focal accommodation. Thus, whereas capsular retention rings may be effective when used in conjunction with non-accommodating lenses, they may prevent premium lenses from functioning properly.

In some cases post surgical adhesions can occur between the lens capsule and the haptic of the intraocular replacement lens. If significant enough, these adhesions can diminish the focal accommodative functions of the lens.

Posterior Capsule Opacification (PCO) is a condition that occurs in approximately fifty percent of cataract patients within three years after surgery. PCO is caused by the migration of epithelial cells from the anterior lens capsule onto and across the posterior capsule, where they can congregate in the form of Elschnig's Pearls or Soemmering's Rings. If the lens epithelial cells migrate to the optical area of the posterior capsule, vision is impaired. The occurrence of PCO can be mitigated surgically by means of Nd-YAG-Laser capsulotomy, which opens a hole in the opacified posterior capsule, restoring clarity of vision. However, Nd-YAG laser capsulotomy surgery also carries risks of post-surgical complications including possible retinal detachment and prolapse of the vitreous into the capsule and anterior chamber. These complications are to be avoided when possible.

IOL designs have found some success at mitigating the onset of PCO by configuring the posterior surface of the lens so as to provide a right angle at the junction of the lens with the posterior capsule. This configuration is particularly applicable for those lenses that rest entirely against the posterior capsule and do not accommodate. Other IOL designs have a surface quality of the haptic may have some influence on PCO mitigation.

There remains a need for an IOL that allows for full accommodation and also reduces or ameliorates the symptoms and causes of PCO.

### SUMMARY OF THE INVENTION

The present invention overcomes the problems and disadvantages associated with current strategies and designs and provides new tools and methods for intraocular lenses and, in particular, haptic devices for intraocular lenses.

One embodiment of the invention is directed to an intracapsular intraocular lens assembly for an eye comprising: a flexible haptic that comprises a continuous ribbon forming an essentially oblong shape having anterior and posterior portions relative to the elliptical center of the haptic, wherein: the ribbon includes two or more congruent ribbon arches in each portion, each ribbon arch has a natural index of curvature with inner and outer edges, and the shape is designed to expand the eye capsule and put tension on the zonules of the eye, and an optic attached to the haptic that is positioned posteriorly to the apex of the congruent ribbon arches and at the center of an optical field of the eye. The lens assembly may be comprised of a hydrophobic, hydrophilic acrylic, or other material that is non-toxic and safe for insertion into the eye, such as, for example a silicone or other similar material know to those skilled in the art. The haptic is essentially oblong shape and is preferably an oval or a rectangular and also preferably, the oval or the rectangular has chamfered or rounded corners. Preferably, the ribbon has dimensions of 1 millimeter or greater in width, or 300 microns or less in depth. Also preferably, the ribbon may have dimensions of 1 millimeter or less in width, or 300 microns or greater in depth. Preferably, the shape retains the natural configuration of the capsule and/or retains the functioning of the zonules and the functional connection between the capsule and the zonules.

Preferably, the optic is suspended on multiple haptic posts, each of which extends from the inner or outer edge of the ribbon and at the elliptical center of the haptic, and wherein the length and width of each haptic post is variable. The optic has a preferred diameter of 5 millimeters or less or 5 millimeters or greater. Preferably each haptic post is rectangular and connected at a right angle to the ribbon loop. Alternatively, haptic pasts may be of all the same or of a variety of shapes such as, for example, trapezoid, square, triangular, and any shape that will function to support the optic. Alternatively, each haptic post may be connected to the ribbon loop at an acute angle and to the optic at an obtuse angle, or to the ribbon loop at an obtuse angle and to the optic at an acute angle. Preferably, the haptic post is connected to the ribbon loop between the inner and outer edges of the ribbon and the ribbon edges are rectangular, and the optic is suspended from the posterior surface of the anterior haptic portion at a distance from the elliptical center of the haptic. Alternatively, ribbon edges may be of other shapes such as, for example, pointed, flattened, beaded, and any other suitable shape. Preferably the apex of the index of curvature is oriented toward the anterior or posterior center or equator of the lens capsule. Preferably two or more ribbon arches are each connected to the perimeter of the lens optic at one or more points. Also preferably, the haptic arches are solid or perforated and, for example, the perforations are in a geometrical, such as for example, a lattice or braid that is rectilinear, curvilinear, geometric or free-form along the ribbon arch, or a random pattern. Preferably the congruent ribbon arches of the anterior portion are essentially perpendicular to the congruent ribbon arches of the posterior portion. Preferably one or more connections between the ribbon arches and the optic comprise one or more hinges. Preferably the anterior and posterior ribbon haptics are connected to each other by means of haptic connection segments for increased stability of positioning within the capsule. Preferably the lens assembly of the invention is configured with optical properties that are refractive, diffractive, spherical or aspherical.

Another embodiment of the invention is directed to an assembly comprising two lens assemblies, each with a haptic and an option, wherein one optic is positioned in the anterior of the capsule and the other optic in the posterior of the capsule, such that each optic is positioned in the optical zone of the eye.

The lens assembly of the invention may further comprise one or more capsular retention rings, e.g. one, two, three or four, that are affixed to one or more points along the congruent ribbon arches or to one or more points along the outer perimeter of the optic. Preferably the retention rings rest against the anterior and posterior capsule of the lens at some distance from the equator, such that the anterior ring arrests the migration of lens epithelial cells along the anterior capsule to the equator, and the posterior ring prevents incursion of PCO in any of its manifestations from the posterior capsular optical zone. Additionally, the retention rings serve to enhance maintaining the natural capsular configuration thus allowing circulation of the aqueous humor throughout the capsule. Preferably the lens assembly of the invention has a retention ring outside the outer perimeter of the optic so as to provide for an ultra-thin optic that risks little to no deformation of the optic during accommodation in the eye.

Another embodiment of the invention is directed to a method of replacing a lens in a patient comprising: removing a lens from an eye of the patient; and replacing the removed lens with a lens assembly of the invention. Preferably the lens assembly provides refractive, diffractive, spherical or aspherical optical properties to the patient and there is little to no deformation of the optic of the lens assembly during accommodation. Preferably the haptic maintain capsular dimension and aperture in all phases of accommodation, wherein, during distance vision, the haptic flattens and moves the optic posteriorly and during close vision, the haptic arches and moves the optic anteriorly. Preferably the lens assembly mitigates migration of epithelial cells and capsular opacification.

Another embodiment of the invention is directed to a method of replacing a lens in a patient comprising removing a lens from an eye of the patient; and replacing the removed lens with a lens assembly of the invention or a conventional lens assembly and an additional lens assembly comprising an additional haptic and an additional optic, wherein one optic is positioned in the anterior of the capsule and the other optic in the posterior of the capsule, such that each optic is positioned in the optical zone of the eye. Preferably during distance vision, the haptic flattens and moves the anterior optic posteriorly, and during close vision, the haptic arches and moves the anterior optic anteriorly. Preferably during close vision, the posterior optic rests against the posterior capsule in an essentially natural configuration.

Another embodiment of the invention comprises an intracapsular intraocular lens assembly for an eye comprising: a flexible anterior haptic that comprises a series of four ribbons forming a cross having anterior and posterior portions relative to the elliptical equator of the natural lens capsule, wherein the ribbon includes two or more congruent ribbon arches in each portion, and each ribbon arch has a natural index of curvature with an inner and outer edges, designed to expand the eye capsule to an essentially natural configuration, such that an essentially natural contact between the capsule, the zonules and the ciliary body is maintained; an optic attached to the anterior haptic and positioned posteriorly to the apex of the congruent ribbon arches; and a flexible posterior haptic that comprises a series of four ribbon in the shape of a cross positioned to rest against the posterior capsule of the eye with the ribbon extending forward and onto the inner surface of the anterior side of the capsule. Preferably the cross of the posterior haptic is positioned at an angle of about 45° or less or more to the cross of the anterior haptic. Preferably the ribbons have ends and the ends are squared, rounded, oval shaped or pointed. Preferably the anterior haptic is connected to the posterior haptic at one or more connection points that maintain a spacing between the two haptics. Preferably a second optic is affixed to the posterior haptic and positioned posteriorly to the apex of the congruent ribbon arches of the posterior haptic.

Another embodiment of the invention is directed toward a haptic configuration comprising four haptic arms that extend from the optic through the capsular equator in a cross form. Preferably the anterior and posterior haptics of this configuration are positioned at a 45° angle from each other and are connected by haptic junctions for stability. Preferably the lens assembly comprises four or more or less haptic arms.

Preferably, the lens assembly further comprising one or more capsular retention rings that are affixed to the anterior haptic, to the posterior haptic or to both haptics. Preferably the one or more capsular retention rings are affixed to one or more points along the congruent ribbon arches or to one or more points along the outer perimeter of the optic. Preferably there is little to no deformation of the optic during accommodation and/or there is a designed deformation of the optic to enhance accommodation. Preferably, the optic is designed to deform so as to provide an increase in accommodation through changes is optical sphericity or asphericity or angle of curvature in refractive or diffractive optic rings.

Other embodiments and advantages of the invention are set forth in part in the description, which follows, and in part, may be obvious from this description, or may be learned from the practice of the invention.

### DESCRIPTION OF THE FIGURES

FIG. 1 depicts the Lens of the invention positioned within the lens capsule as looking at the lens inward from the center of the eye towards the retina. In this drawing the lens optic is described as having a 5 millimeter diameter, and is suspended posteriorly toward the retina from the ribbon haptic as shown. The anterior portion of the haptic arches will expand the anterior capsule and the posterior portion will expand the posterior capsule posteriorly away from the anterior zonules, thus placing tension on both the anterior and posterior zonules in the accommodative state. The general diameter of the capsule at the prime meridian, or equator, is measured at 8.9 millimeters in the accommodative state.

FIG. 2 depicts the cross section of the Lens of the invention in the accommodative, or near vision state, illustrating the extension of the ribbon arced haptic from the anterior capsule through the lens equator and to the posterior capsule some distance from the center. The positioning of the lens optic, and the angle and relative dimensions of the haptic bridges connecting to the optic are also shown.

FIG. 3 depicts a detail of the Lens of the invention in the accommodative state, articulating the capsular extension quality of the haptic arch, preserving as much as possible the natural curvature of the lens capsule. The lens optic is shown suspended from the anterior haptic ribbon. The end point of the posterior haptic arc is shown on the posterior capsule outside of the imputed diameter of the optic, preserving clear optical light transfer.

FIG. 4 depicts a detail of the Lens of the invention in the non-accommodative, or distance vision state, articulating the dynamic elongation of the capsule at the equator from an imputed accommodative diameter of 8.9 millimeters to an imputed diameter of 9.6 millimeters. In this detail, the inventive haptic arc is flexed at the lens equator to a smaller imputed radius of curvature, responding to the relaxation or retraction of the ciliary body and concomitant pulling of the zonules on the capsule, which naturally deforms the capsule to the distance vision state. This figure also demonstrates the position of the lens optic in the capsule, which has been moved toward the posterior capsular surface in response to the haptic flex.

FIG. 5 depicts the posterior haptic arch of the second haptic of the invention, showing its position at a right angle to the longitudinal meridian of the anterior haptic. This figure also demonstrates the plano disc protecting the optical region of the posterior capsule. This figure demonstrates the lens in the accommodative (near vision) state.

FIG. 6 depicts the posterior haptic arch in the distance vision position. This figure also incorporates optional anchor lines from the anterior to the posterior haptic arches, preserving proper positioning and centration of the lens optic in the eye, and suitable separation of the two haptic pieces.

FIG. 7 illustrates an alternative haptic connection arm to the optic, describing an arched ribbon haptic in the accommodative state. This figure also demonstrates a hinge mechanism connecting the haptic to the lens optic.

FIG. 8 illustrates the arched ribbon haptic connection arm in the distance vision state.

FIG. 9 illustrates two alternative haptic ribbon arches from each anterior haptic ribbon connection point to the lens optic, (a) for near vision and for (b) distance vision.

FIG. 10 illustrates sections of the arched ribbon haptic in cross section view showing open spaces in the haptic ribbon.

FIG. 11 illustrates the arched ribbon haptic in other configurations: (a) with the curvature anterior and towards the pupil; (b) with the curvature anterior and towards the equator; (c) with the curvature posterior towards the retina; and (d) with the curvature posterior toward the vitreous humor.

FIG. 12 illustrates an optic retention ring for preventing deformation of the optic in accommodation.

FIG. 13 illustrates indicative placement in a longitudinal view (a) and in a cross sectional view (b) of capsular retention rings to the anterior and posterior haptic ribbons for the purpose of arresting epithelial cell migration on the anterior capsule and intrusion of PCO into the optic zone of the posterior capsule.

FIG. 14 depicts placement of the haptic and optic in the eye capsule whereby; (a) the haptic extends posteriorly past the lens equator; and (b) the haptic extends anteriorly past the lens equator.

FIG. 15 depicts placement of the optic and haptic in the eye capsule with a ribbon haptic structure containing both anterior and posterior haptic retention rings and a posterior plate. Also depicted is a haptic configuration of four haptic arms each, anterior and posterior, that extend from the relevant optic in the form of a cross and continue through the meridian of the lens, with the anterior haptic arm positioned 45° from posterior haptic arm.

FIG. 16 depicts (a) an anterior view and (b) a posterior view, of the haptic and optic of FIG. 15.

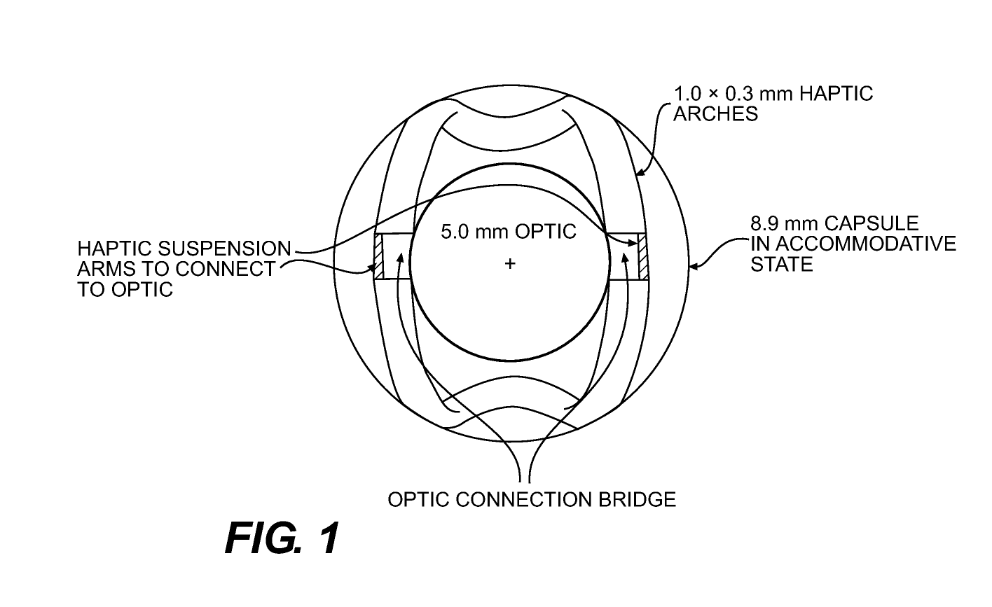

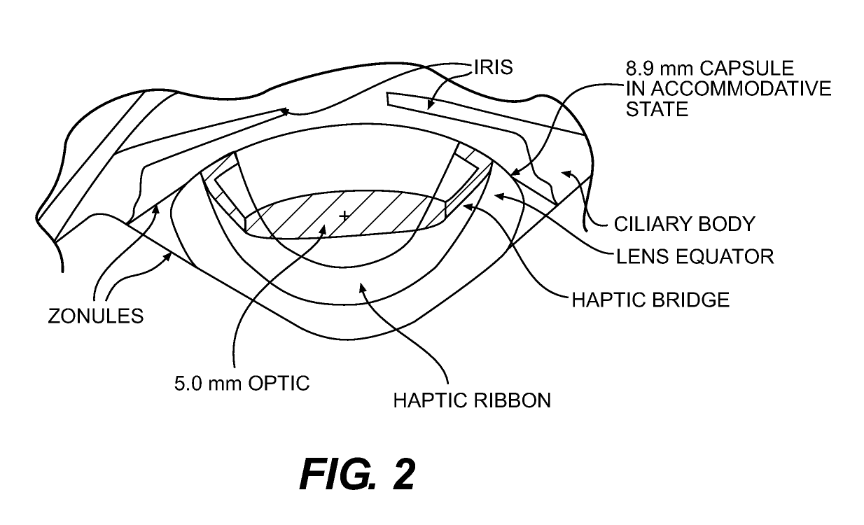

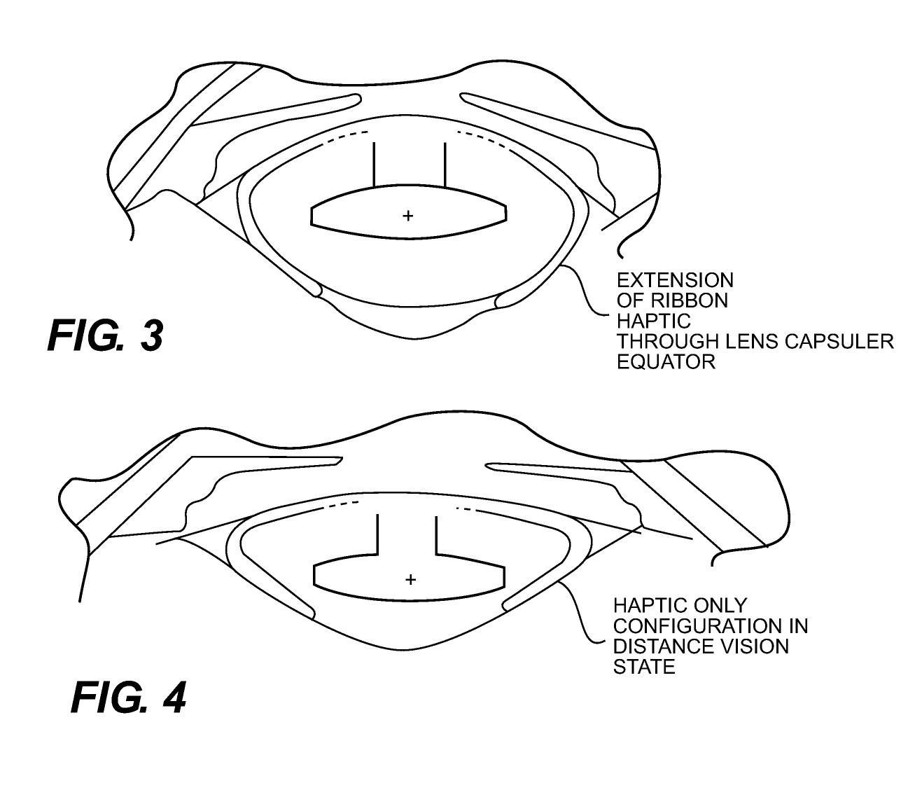

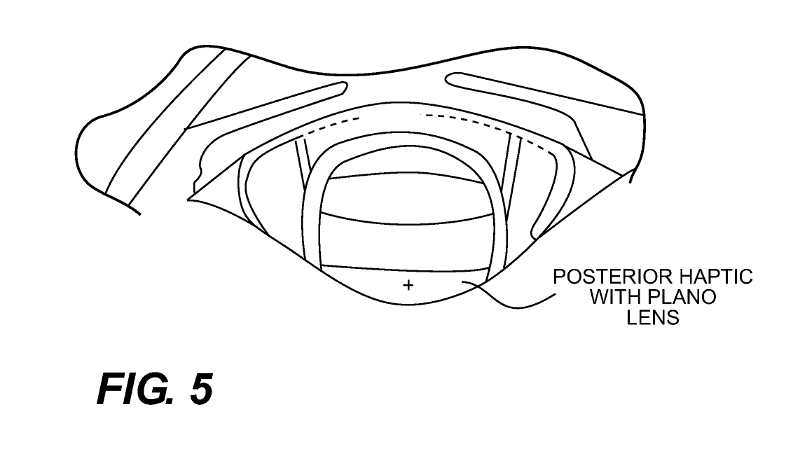

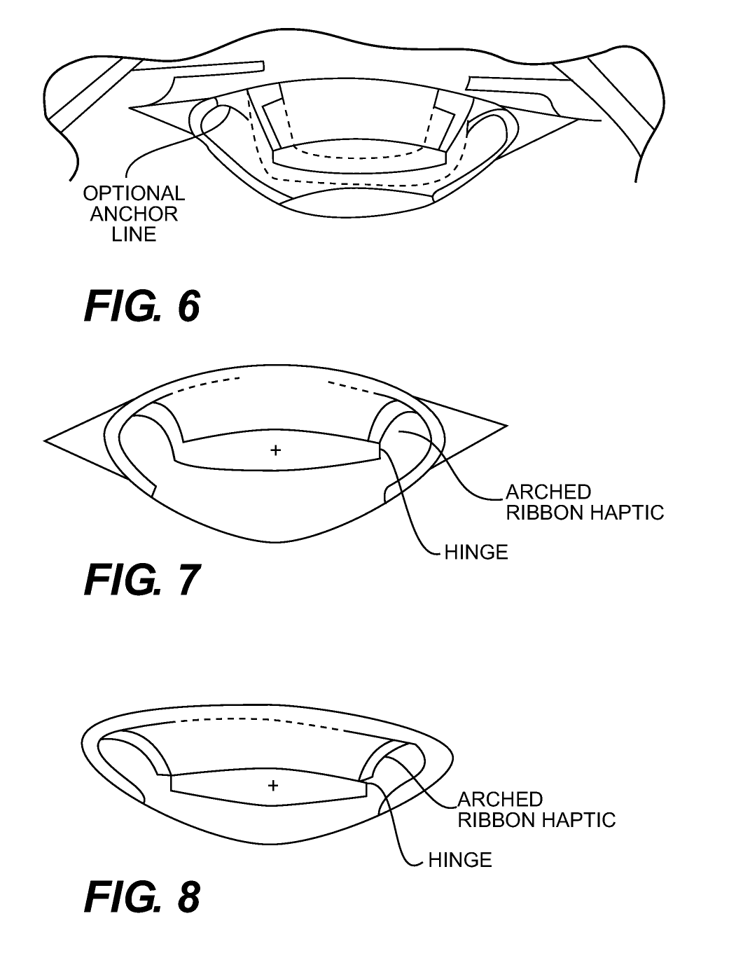

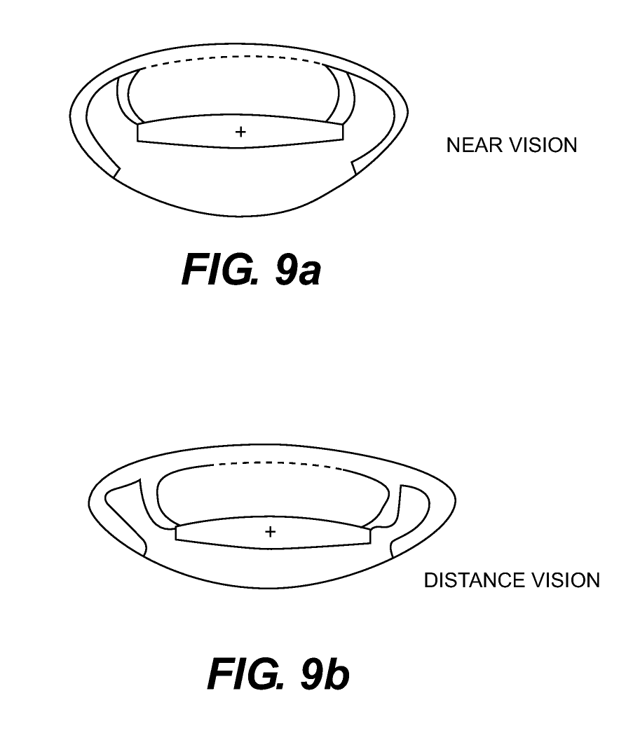

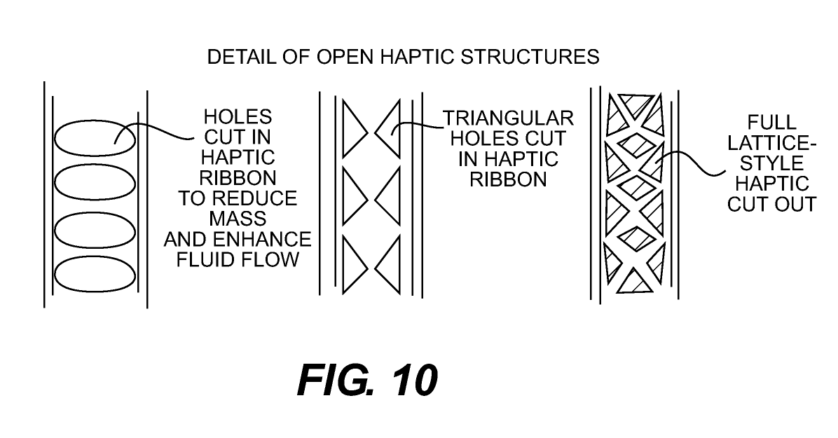

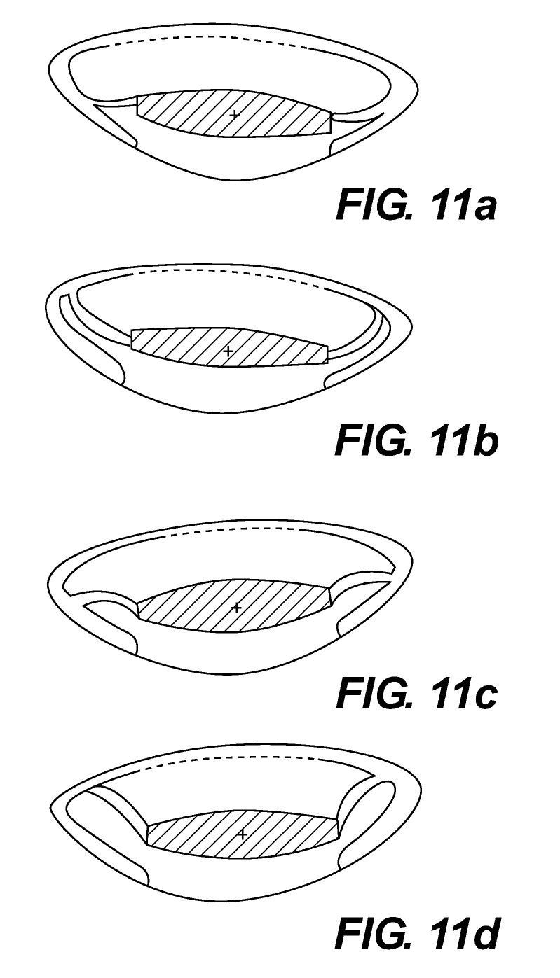

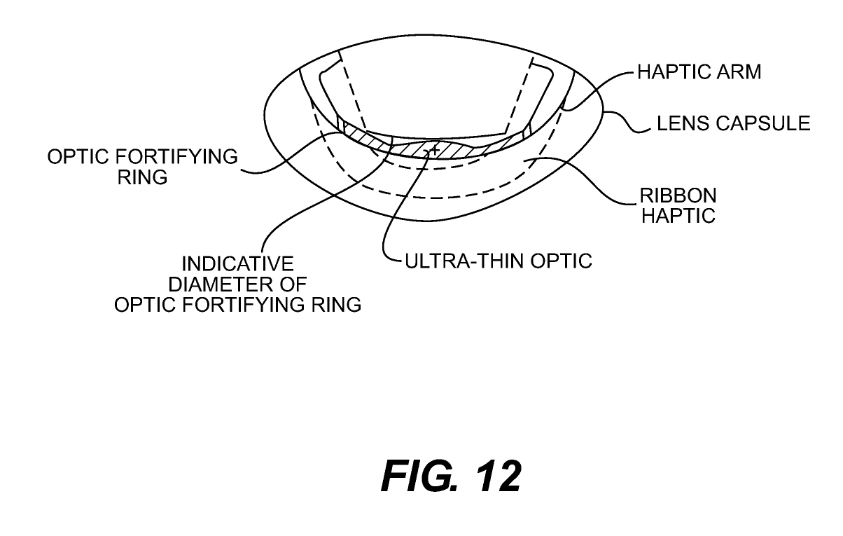

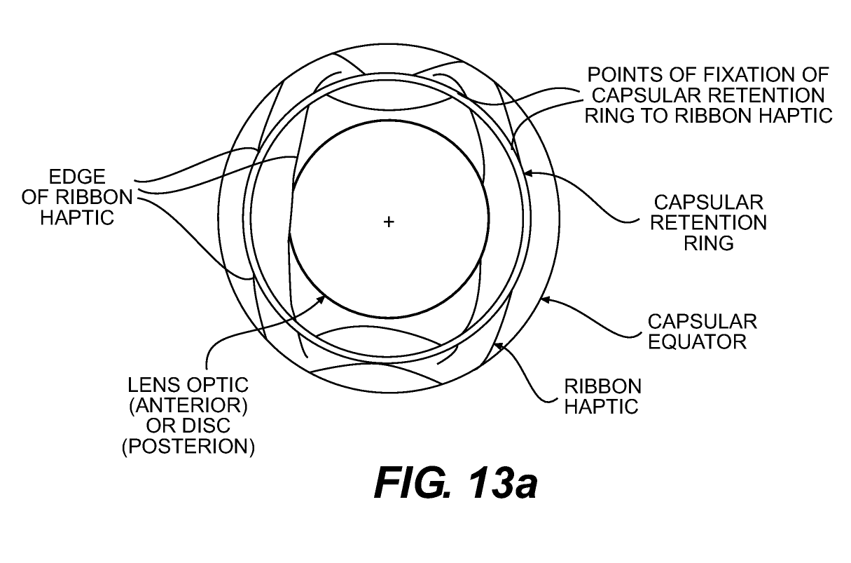

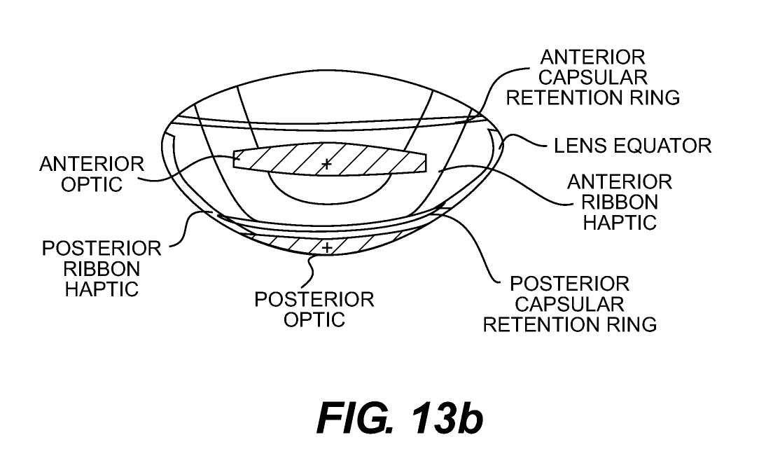

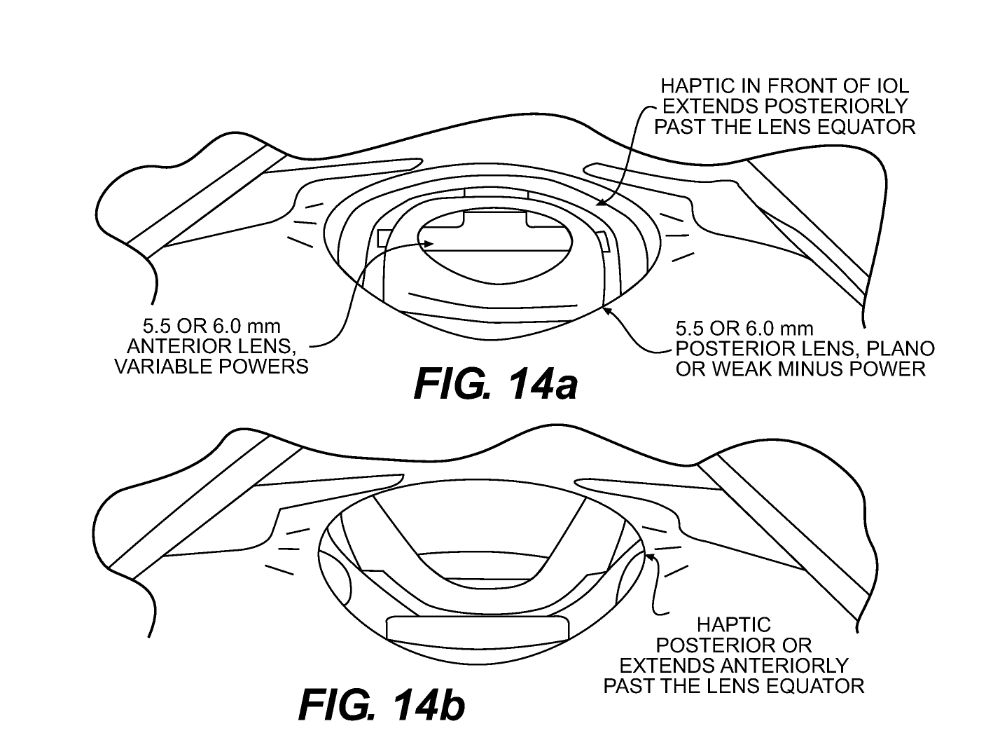

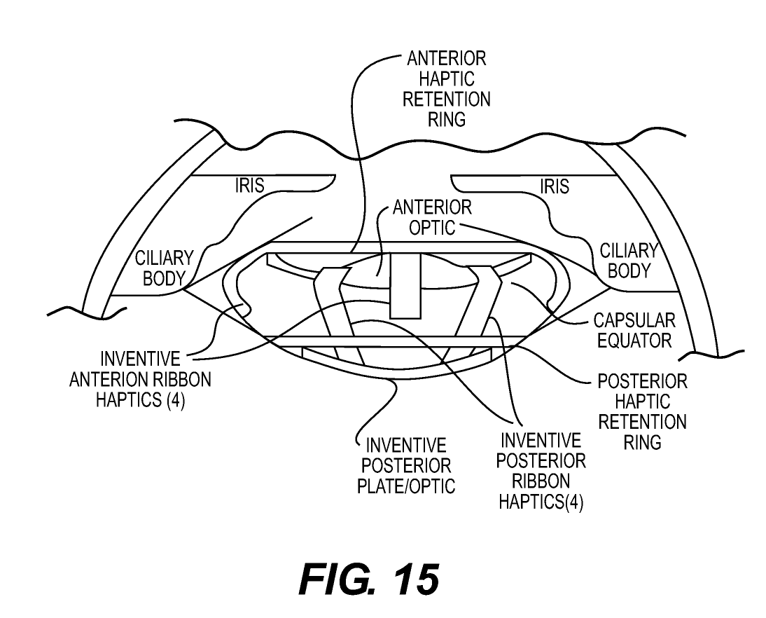

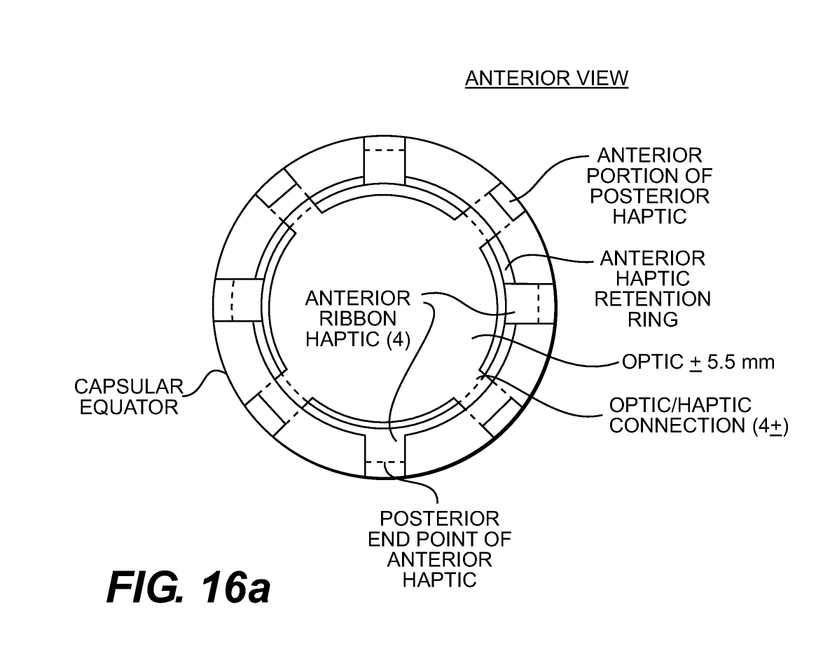

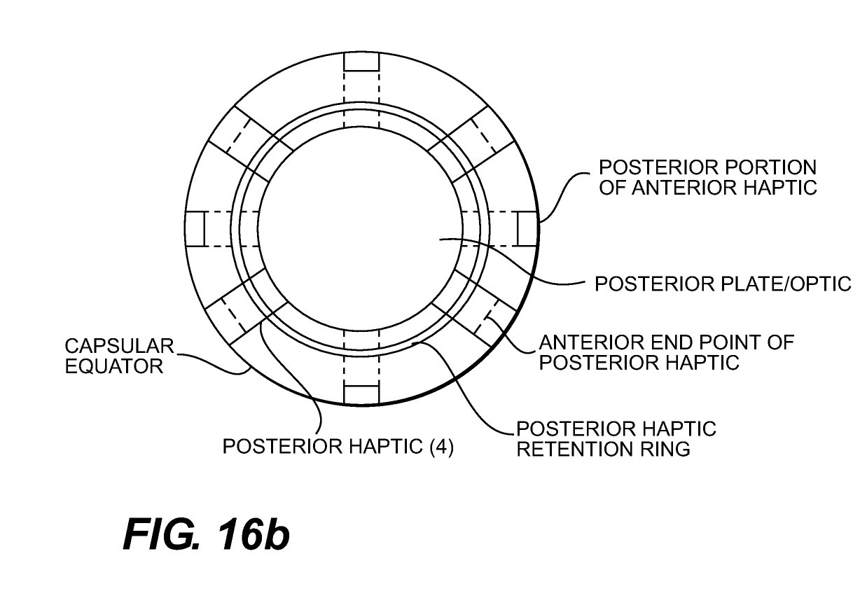

### DESCRIPTION OF THE INVENTION

The invention is directed to a haptic for fixation to, and manufacture in conjunction with, an intraocular lens to be implanted in the natural lens capsule of the human eye, once the natural crystalline lens has been surgically removed. The function of the haptic is to secure the lens in an appropriate position within the natural capsule so as to provide optimal visual acuity through the aphakic lens. The haptic is designed to affix to the lens on each side of the optic edge at a point or a series of points so as to provide suitable centration and stability of the optic, and so as to suspend the optic in the open capsular space. The haptic arm is a band of the haptic material that extends from the optical connection to connect with a solid ribbon of haptic material forming a constant loop across the anterior capsule, across the capsular prime meridian, or equator, and onto the posterior capsular surface, terminating at a point distally outward from the optical zone on the posterior capsule. The material of the haptic is preferably flexible and elastic, so as to provide a constant, positive force on the capsule throughout all phases of accommodation, thereby preserving tension of the zonules and allowing the capsule to change shape naturally. The haptic ribbons may be solid or of an open work structure to increase the amount of hydration available to the lens capsule. A secondary haptic ribbon, affixed to a plano optical plate, is located on the posterior capsular surface and oriented so that the haptic arms extend through the capsular prime meridian to the anterior capsular surface at a 90° angle from the anterior haptic ribbons, thus providing for a capsular configuration as natural as possible, yet associated with an intraocular lens that may be inserted through an incision of less than 3 millimeters. The suspended optic will move in the eye in response to the motion of the ciliary body and attendant tension of the zonules, providing focal accommodation. The optic on the posterior haptic enhances the accommodative effect and prevents the aggregation of any PCO in the optical zone of the posterior capsule but also serves to prevent possible prolapse of the vitreous humor in the event a posterior capsulotomy were performed. Additionally, the design of the haptic inhibits the migration of epithelial cells from the anterior to the posterior capsule, thereby mitigating Posterior Capsular Opacification (PCO). As part of the IOL of the invention, capsular retention rings may be affixed to the ribbon haptic to be positioned against the surface of the lens capsule at locations both anterior and posterior to the lens capsular equator so as to create a fully circular impediment to epithelial cell migration along the interior capsule to the lens equator, and of resultant PCO along the posterior capsule into the optic zone. In another embodiment, the arms of the haptic are modestly arched to increase focal flexibility, and may be affixed to the anterior haptic ribbon at some distance from the apex of the anterior capsule. Preferably, the haptic ends are designed to position the lens neutrally, anteriorly or posteriorly within the lens envelope. Preferably, the anterior and posterior haptics are joined together by small connecting segments of haptic material to preserve relative position of the two haptics within the eye. The haptic design facilitates compressing the lens into its injector for insertion into the eye through an incision in the cornea and/or sclera. Once compressed and passed through the corneal, corneoscleral or scleral incision, the implanted lens is secured by the haptics in the lens capsule once all possible natural lens material and epithelial cells have been removed.

In the case of the inventive haptic designs discussed herein, the onset of PCO may be delayed or eliminated altogether through the use of appropriate haptic design to deter epithelial cell migration. In particular, 1) a haptic design that keeps the capsule open and prevents contact between the anterior and posterior surfaces assist in mitigating PCO onset by maintaining hydration of the capsule and circulation of the aqueous humor, 2) the quality of the cataract or CLEAR surgery assists in retarding PCO through assiduous cleaning and polishing of the anterior capsule, 3) the secondary haptic band with its disc protecting the posterior capsular surface directly in the optical zone maintains clarity of vision and prevent vitreous prolapse in the event of a Nd-YAG laser capsulotomy, 4) suitably formulated edge structures placed against the surface of the posterior capsule arrests the migration of epithelial cells and prevent their aggregation in the posterior capsular optic zone, and 5) retention rings on the anterior and posterior capsular surface block epithelial cell migration and maintain a clear posterior optic zone. In another embodiment, a secondary haptic is configured with a plano surface or either positive or negative optical powers. Preferably the anterior surface is formed as a sphere, an asphere, a toric design, or a series of refractive or diffractive steps so as to enhance the accommodative effect of the inventive lens and haptic.

One embodiment of the invention is directed to an intracapsular intraocular lens comprising a haptic and an optic. The haptic of the invention preferably comprises a flexible acrylic ribbon in a loop that is preferably oblong in shape, with a natural index of curvature designed to maintain or even to expand the capsule and, also preferably, put tension on the zonules, and a monofocal optic suspended posteriorly to the apex of the ribbon arch and at the center of the eye's optical field. The haptic of the invention maintains an even pressure throughout the capsule, keeping the capsule open and hydrated, and serves as a plug to keep vitreous fluid within the capsule. Preferably, one face of the ribbon materials is maintained against the walls of the capsule, preferably maintaining a relatively spherical and natural cavity. Further, the haptic of the invention preferably allows for the incorporation of most any optic, including a negatively optic lens, and for correction of astigmatism in an accommodating lens. Preferably, the posterior optic is configured so as to be placed as close to the nodal zone of the eye as possible to provide maximum depth of field.

Another embodiment of the invention incorporates a capsular ring with the haptic of the invention to further support the capsule cavity and preferably allows for the use of relatively thin optics.

Another embodiment of the invention is directed to the haptic of the invention coupled with a second haptic, approximately perpendicular to the first haptic. The coupled haptics preferably provide for an open and hydrated capsular space and maximize accommodative effect.

The lens of the invention is an intra-capsular intraocular lens comprising a flexible loop (e.g. acrylic) that is sized to fit against the inside of the natural lens capsule across the anterior capsule, through the prime meridian or equator of the lens capsule and to a point on the posterior capsule distally outward from that central portion of the posterior capsule directly and having an optic of at least five millimeters. The haptic loop of the lens of the invention is formed as a ribbon, preferably one millimeter wide and 300 microns thick, with a natural curvature to the haptic ideally to conform to the natural curvature of the natural, crystalline lens in the accommodative, or near vision, state. The design of the lens haptics is to maintain separation of the anterior and posterior segments of the lens capsule, allowing the capsule and the lens to maintain their proper position and function, thus ensuring preservation of the proper refractive state and the accommodative effectiveness. Thus the haptic maintains capsular dimension and aperture in all phases of accommodation. The haptic loop will respond to the natural tension of the zonules on the lens capsule in the distant vision state, and flatten somewhat, thus exercising posterior thrust on the lens optic that is centrally suspended from the anterior haptic arms. Preferably the optic is positioned so as to be located on a plane that is anterior to the equator of the lens capsule in the accommodative state, and to be located on a plane posterior to the equator of the lens capsule in the distance vision state. The accommodative method of the haptic is to respond to the relaxation of the ciliary body as it moves outward for distance vision, which increases outward tension on the zonules, thus compressing the haptic arch and moving the optic posteriorly. Conversely, as the ciliary body moves anteriorly during accommodative effort, the haptic arches reconfigure the lens capsule to a more spherical shape, with the anterior capsule of the lens in close proximity to the iris, which moves the optic anteriorly in both the lens capsule and the eye.

The lens assembly of the invention also preferably comprises a posterior haptic and optic, positioned in the eye at the same time as the anterior haptic and optic, and positioned such that the posterior haptic ribbons are placed at a right angle to the anterior haptic ribbons, thus maintaining as much as possible the natural aperture and configuration of the lens capsule. The posterior haptic is designed with a lens optic that extends directly from the haptic ribbons and rests securely against the posterior capsule. Preferably, this optic is structured so as to be plano, thus providing no additional optical power but serving to protect the optical area of the posterior capsule. Preferably, the posterior optic is engineered to provide optical power, positive or negative, toric, refractive, or diffractive so as to enhance the accommodative effect of the anterior optic and work in harmony with the anterior optic in its accommodative response. The position of the posterior optic also increases depth of field in the patient, enhancing visual acuity and range of accommodation.

Another variation in the design of the lens of the invention includes one or more rings, preferably one, two or three, affixed on the same plane as the ribbon haptic, either anterior and posterior or both, in all cases at some distance from the natural lens equator, such that epithelial cell migration and/or progression of PCO is arrested at the location of such rings, thus preserving a larger open optical area. These rings are preferably made of the same material as the haptic, and may also be affixed to the lens prior to insertion into the eye.

The materials applicable to the lens assembly of the invention may comprise hydrophilic acrylic, hydrophobic acrylic, silicone or other suitable, flexible material, approved for intraocular use, and such that it retains sufficient molecular memory to provide for constant positioning of the lens against the inner capsular wall. Moreover the acrylic material is flexible enough to change shape easily and respond to the prompts of the ciliary body, but resilient or sufficiently stiff to resist cracking or other deterioration for decades. The constant contact of the haptic ribbon with the lens capsule is an important factor in mitigating the migration of epithelial cells along the anterior capsule to the equator, which is the cause of Posterior Capsular Opacification (or PCO) in many post-cataract surgery patients. Preferably, the surface of the one millimeter planes of the haptic ribbon is perpendicular to the 300 micron planes so as to nestle snugly against the capsule and provide rectangular edge, which further restricts epithelial cell migration. Preferably the haptic design of the invention maintains the lens capsule open, thus preventing adhesions between the anterior and posterior surfaces of the capsule. Maintaining the capsule open allows the aqueous humor to circulate within the capsule, which provides for enhanced hydration of the lens capsule over models of intraocular lenses that are primarily two-dimensional in their configuration and which stretch the lens capsule out horizontally. Another preferred feature of the lens of the invention is that it adjusts to fit a wide variety of lens capsule sizes and shapes. All human lens capsules are not identical in circumference or volume, which means that certain intraocular lenses will not fit certain patients, and also that a lens that does fit at the time of the lens replacement surgery may cease to fit properly in the event of capsular atrophy or adhesions due to, amongst other possible causes, contact between the lens capsular surfaces, dehydration of certain areas of the lens capsule as a result of insufficient aqueous humor circulation, or PCO, specifically in the manifestation of Elschnig's Pearls or Soemmering's Rings. The lens of the invention, with its ribbon haptic design, preferably adjusts to fit a wide range of eyes, the limiting factor being the distance between the end points of the haptic loops on the posterior capsular surface. Moreover, the elastic pressure of the haptic of the invention exerts positive influence on the capsule, encouraging prolonged elasticity and curbing capsular contraction tendencies.

In another preferred embodiment of the invention, a second ribbon haptic mechanism is inserted in an inverse position resting against the posterior capsule, with the haptic ribbon arms extending through the capsular equator and onto the inner face of the anterior capsule. An element of the secondary lens is to provide a fuller, that is to say, spherical configuration to the lens capsule, to provide increased damming qualities against epithelial cell migration, and to maintain the optical portion of the posterior capsule free from threats of PCO. There is another key use for the second haptic mechanism, which is that, in the event that the ophthalmologist determines to execute a Nd-YAG Laser Capsulotomy, the second optical piece, which by design is a plano or negatively powered lens, will serve as a permanent protection against possible prolapse of the vitreous into the lens capsule and the anterior chamber which is a potential hazard of any posterior capsulotomy. In addition, the second optic increases depth of field.

A variation of the second ribbon haptic mechanism contemplates a flexible connection between anterior and posterior haptic segments such that the anterior and posterior haptics are fixed at 90° to each other but with sufficient flexibility to allow the haptics to move closer to or farther away from each other as the configuration of the lens capsule changes through the accommodative process. A purpose of this invention is to preserve the stability of the geometrical proportion of the two haptic structures while being as responsive as possible to the natural movement of the lens capsule through accommodation. A novelty of this approach is to provide an overall lens structure and design that is capable of being inserted into the eye through an incision of less than 3 millimeters, thus requiring no sutures, but providing constant and elastic support to the entire lens capsule, thus maintaining as much as possible the same configuration of the eye as existed prior to the removal of the natural, crystalline lens. This inventive configuration provides the opportunity that the lens may be inserted in a younger patient than the normal cataract patient, using the CLEAR procedure, as preserving natural lens shape and configuration is important to providing the right environment for a presbyopia correcting lens. Additionally, keeping the lens capsule open prolongs the useful life of the Lens of the invention as the capsule can remain hydrated by the aqueous humor, which prolongs and/or prevent the onset of capsular shrinkage and adhesions.

In another embodiment of the haptic of the invention, the ribbon haptics preferably contains a series of perforations so as to increase the percentage of the lens capsule accessible to the natural hydration and circulation of the aqueous humor. A haptic ribbon, thus, may be solid, may be scored with perforations, may contain a lattice-like structure, or any variations thereof, still preserving the elastic functionality of the haptic arms so as to meet the desired accommodative objectives of the Lens of the invention. This, over time, provides for certain design features of the haptic of the invention that are particularly applicable to different types of patient, whether defined by age, race, gender, medical condition, or other criteria as a competent ophthalmologist may determine.

The assembly of the invention incorporates an optic with a diameter of 5 mm that is suspended from the anterior ribbon by means of two posteriorly oriented arms that extend from the outer perimeter of the ribbon and measure approximately 1.5 mm in length and up to 350 microns in width. These arms then connect to the outer edge of the optic. The length of the arms may vary as to the specific needs of the patient, the optical powers required in the accommodative process, and other factors as the ophthalmologist may determine. The optic may be configured as a spherical, aspherical, refractive, diffractive optic, such as the diopter power of the lens may require, with any blend of such optical styles as between the anterior and posterior surface of the lens. Because the Lens of the invention optic is suspended in the center of the capsular space, the optic surface will not come into contact with the capsule at any time. By contrast, the posterior Lens of the invention haptic ribbon connects directly to the plano optical center such that this center is in contact with the center of the posterior capsule. This mechanism protects the posterior capsule from PCO, and obviates the need for a posterior capsulotomy, thereby protecting the integrity of the lens capsule and minimizing the risk of vitreous prolapse.

In another embodiment of the lens, the optic is preferably centrally suspended from the haptic ribbon by means of an arced segment that originates at the haptic arm at a point distally outward from the circumference of the optic and distally inward from the point at which the haptic arm contacts the prime meridian of the lens capsule. The arced segment comprises a tapered ribbon narrowest at its connection point to the optic, and may or may not be hinged at the optical point of contact. The orientation of this ribbon is geometrically perpendicular to that of the haptic ribbon, that is to say, with the broader expanse of the ribbon oriented anteriorly and posteriorly in the lens capsule so as to provide support for the lens movement within the capsule through the accommodative process. In another embodiment the arced segments is number two or three at each connection point to the optic thereby providing for consistent centration and orientation of the lens optic at all times. In all cases, these arced segments may be solid, or may have an open work construction similar to the flying buttresses of a gothic cathedral. In another preferred embodiment, the arced segments may connect at various points along the circumference of the optic. In any or all of these embodiments, the diameter of the optic may be increased to greater than 5.5 millimeters.

Arced segments connecting the lens optic to the haptic are beneficial, especially if the lens optic is an ultra-thin diffractive or refractive optic, to prevent deformation of the outer portion of the optic in the accommodative process by means of a slightly thicker ring attached to and positioned immediately at the outer edge of the optic. This ring also provides a substantially sturdier connection point for the arced segments and allows for the addition of hinges to further increase motion of the optic in accommodation.

Other embodiments and uses of the invention will be apparent to those skilled in the art from consideration of the specification and practice of the invention disclosed herein. All references cited herein, including all publications, U.S. and foreign patents and patent applications and priority documents (U.S. Provisional Application No. 61/347,083, U.S. Provisional Application No. 61/381,784, U.S. Provisional Application No. 61/118,076, U.S. Provisional Application No. 61/118,085, U.S. application Ser. No. 12/626,459, U.S. application Ser. No. 12/626,473), are specifically and entirely incorporated by reference. The term comprising, where ever used, is intended to include the terms consisting and consisting essentially of. Furthermore, the terms comprising, including, and containing are not intended to be limiting. It is intended that the specification and examples be considered exemplary only with the true scope and spirit of the invention indicated by the following claims.

1. An intracapsular intraocular lens assembly for an eye comprising:
a flexible haptic that comprises a continuous ribbon forming an essentially oblong shape having anterior and posterior portions relative to the elliptical center of the haptic, wherein:
the ribbon includes two or more congruent ribbon arches in each portion,
each ribbon arch has a natural index of curvature with inner and outer edges, and
the shape is designed to expand the eye capsule and put tension on the zonules of the eye, and
an optic attached to the haptic that is positioned posteriorly to the apex of the congruent ribbon arches and at the center of an optical field of the eye.

2. The lens assembly of claim 1, which is comprised of a hydrophobic, hydrophilic or acrylic material that is non-toxic and safe for insertion into the eye.

3. The lens assembly of claim 2, wherein the material is a silicone.

4. The lens assembly of claim 1, wherein the essentially oblong shape is an oval or a rectangle.

5. The lens assembly of claim 4, wherein the oval or the rectangle has chamfered or rounded corners.

6. The lens assembly of claim 1, wherein the ribbon has dimensions of 1 millimeter or greater in width, or 300 microns or less in depth.

7. The lens assembly of claim 1, wherein the ribbon has dimensions of 1 millimeter or less in width, or 300 microns or greater in depth.

8. The lens assembly of claim 1, wherein expansion of the eye capsule retains a natural configuration of the eye capsule or retains the functional connection between the eye capsule and the zonules of the eye.

9. The lens assembly of claim 1, wherein the optic is suspended on multiple haptic posts, each of which extends from the inner or outer edge of the ribbon and at the elliptical center of the haptic, and wherein the length and width of each haptic post is variable.

10. The lens assembly of claim 9, wherein the optic has a diameter of 5 millimeters or less.

11. The lens assembly of claim 9, wherein the optic has a diameter of 5 millimeters or greater.

12. The lens assembly of claim 9, wherein each haptic post is rectangular and connected at a right angle to the ribbon loop.

13. The lens assembly of claim 9, wherein each haptic post is connected to the ribbon loop at an acute angle and to the optic at an obtuse angle.

14. The lens assembly of claim 9, wherein the haptic post is connected to the ribbon loop at an obtuse angle and to the optic at an acute angle.

15. The lens assembly of claim 9, wherein the haptic post is connected to the ribbon loop between the inner and outer edges of the ribbon and the ribbon edges are rectangular.

16. The lens assembly of claim 1, wherein the optic is suspended from the posterior surface of the anterior haptic portion at a distance from the elliptical center of the haptic.

17. The lens assembly of claim 1, wherein the apex of the index of curvature is oriented toward the anterior or posterior center of the lens capsule.

18. The lens assembly of claim 1, wherein the apex of the index of curvature is oriented toward the anterior or posterior equator of the lens capsule.

19. The lens assembly of claim 1, wherein two or more ribbon arches are each connected to the perimeter of the lens optic at one or more points.

20. The lens assembly of claim 1, wherein the haptic arches are solid or perforated.

21. The lens assembly of claim 20, wherein the perforations are in a geometrical or random pattern.

22. The lens assembly of claim 21, wherein the geometrical pattern is a lattice or braid that is rectilinear, curvilinear, geometric or free-form along the ribbon arch.

23. The lens assembly of claim 1, wherein the congruent ribbon arches of the anterior portion are essentially perpendicular to the congruent ribbon arches of the posterior portion.

24. The lens assembly of claim 1, wherein one or more connections between the ribbon arches and the optic comprises one or more hinges.

25. The lens assembly of claim 1, which is configured with optical properties that are refractive, diffractive, spherical or aspherical.

26. The lens assembly of claim 1, further comprising an additional lens assembly comprising an additional haptic and an additional optic, wherein one optic is positioned in the anterior of the capsule and the other optic in the posterior of the capsule, such that each optic is positioned in the optical zone of the eye.

27. The lens assembly of claim 1, further comprising one or more capsular retention rings that are affixed to one or more points along the congruent ribbon arches or to one or more points along the outer perimeter of the optic.

28. The lens assembly of claim 1, wherein there is little to no deformation of the optic during accommodation.

29. The lens assembly of claim 1, wherein there is a designed deformation of the optic to enhance accommodation

30. A method of replacing a lens in a patient comprising:
removing a lens from an eye of the patient;
replacing the removed lens with the lens assembly of claim 1.

31. The method of claim 30, wherein the lens assembly provides refractive, diffractive, spherical or aspherical optical properties to the patient.

32. The method of claim 30, wherein there is little to no deformation of the optic of the lens assembly during accommodation.

33. The method of claim 30, wherein the haptic maintain capsular dimension and aperture in all phases of accommodation.

34. The method of claim 30, wherein, during distance vision, the haptic flattens and moves the optic posteriorly.

35. The method of claim 30, wherein, during close vision, the haptic arches and moves the optic anteriorly.

36. The method of claim 30, wherein the lens assembly mitigates migration of epithelial cells and capsular opacification.

37. A method of replacing a lens in a patient comprising:
removing a lens from an eye of the patient;
replacing the removed lens with the lens assembly of claim 26.

38. The method of claim 37, wherein, during distance vision, the haptic flattens and moves the anterior optic posteriorly.

39. The method of claim 37, wherein, during close vision, the haptic arches and moves the anterior optic anteriorly.

40. The method of claim 37, wherein, during close vision, the posterior optic rests against the posterior capsule in an essentially natural configuration.

41. An intracapsular intraocular lens assembly for an eye comprising:
a flexible anterior haptic that comprises a series of four ribbons forming a cross having anterior and posterior portions relative to the elliptical equator of the natural lens capsule, wherein the ribbon includes two or more congruent ribbon arches in each portion, and each ribbon arch has a natural index of curvature with an inner and outer edges, designed to expand the eye capsule to an essentially natural configuration;
an optic attached to the anterior haptic and positioned posteriorly to the apex of the congruent ribbon arches; and
a flexible posterior haptic that comprises a series of four ribbon in the shape of a cross positioned to rest against the posterior capsule of the eye with the ribbon extending forward and onto the inner surface of the anterior side of the capsule.

42. The lens assembly of claim 41, wherein the cross of the posterior haptic is positioned at an angle of about 45° or less to the cross of the anterior haptic.

43. The lens assembly of claim 41, wherein the cross of the posterior haptic is positioned at an angle of about 45° or more to the cross of the anterior haptic

44. The lens assembly of claim 41, wherein the ribbons have ends and the ends are squared, rounded, oval shaped or pointed.

45. The lens assembly of claims 41, where the anterior haptic is connected to the posterior haptic at one or more connection points that maintain a spacing between the two haptics.

46. The lens assembly of claim 41, further comprising another optic attached to the posterior haptic and positioned posteriorly to the apex of the congruent ribbon arches of the posterior haptic.

47. The lens assembly of claim 41, further comprising one or more capsular retention rings that are affixed to the anterior haptic, to the posterior haptic or to both haptics.

48. The lens assembly of claim 41, further comprising one or more capsular retention rings that are affixed to one or more points along the congruent ribbon arches or to one or more points along the outer perimeter of the optic.

49. The lens assembly of claim 41, wherein there is little to no deformation of the optic during accommodation.

50. The lens assembly of claim 41, wherein there is a designed deformation of the optic to enhance accommodation

51. The lens assembly of claim 41, wherein there are more or less than four haptic arms.

52. A method of replacing a lens in a patient comprising:
removing a lens from an eye of the patient;
replacing the removed lens with the lens assembly of claim 41.
